(19) United States
(12) Patent Application Publication
(10) Pub. No.: US 2024/0024281 A1
(43) Pub. Date: Jan. 25, 2024

(54) METHODS AND COMPOSITIONS FOR TREATING LIVER DISEASES

(71) Applicants: INSTITUT NATIONAL DE LA SANTÉ ET DE LA RECHERCHE MÉDICALE, Paris (FR); CENTRE HOSPITALIER UNIVERSITAIRE DE NICE, Nice (FR); UNIVERSITE COTE D'AZUR, Nice (FR)

(72) Inventors: Beatrice BAILLY-MAITRE RE, Nice Cedex 3 (FR); Philippe GUAL, Nice Cedex 3 (FR); Albert TRAN, Nice Cedex 3 (FR)

(21) Appl. No.: 18/328,042

(22) Filed: Jun. 2, 2023

Related U.S. Application Data
(62) Division of application No. 16/970,031, filed on Aug. 14, 2020, now abandoned.

(30) Foreign Application Priority Data
Feb. 16, 2018 (EP) 18305169.7

(51) Int. Cl. A61K 31/381; A61P 1/16; A61P 35/00

(52) U.S. Cl. CPC A61K 31/381; A61P 1/16; A61P 35/00

(57) ABSTRACT

The present invention relates to a method for treating a subject suffering from a liver disease comprising a step of administering said subject with a therapeutically effective amount of an inhibitor of the endoribonuclease activity of IRE1α. Inventors have shown that in livers of tunicamycin-treated BI-1−/− mice aIRE1α-dependent NLRP3 inflammasome activation, an hepatocyte death, a fibrosis and a dysregulated lipid homeostasis that led to liver failure within a week. To test whether the pharmacological inhibition of IRE1α endoribonuclease activity would block the transition to NASH, mice were injected with the small molecule STF-083010 twice a week for 2 weeks towards the end of a 3-month HFD. In BI-1−/− mice, STF-083010 treatment effectively counteracted IRE1α endoribonuclease activity, improving glucose tolerance and rescuing from NASH. The hepatocyte-specific role of IRE1α's RNase activity in mediating NLRP3 inflammasome activation and programmed cell death was confirmed in primary mouse hepatocytes through knockdown experiments and with STF-083010.

### FIELD OF THE INVENTION

The invention is in the field of hepatology. More particularly, the invention relates to method and composition for treating liver diseases.

### BACKGROUND OF THE INVENTION

Obesity with its comorbidities constitutes an authentic pandemia with catastrophic consequences for public health. The global prevalence of non-alcoholic fatty liver disease (NAFLD) is estimated to be >25%¹. This hepatic pathology, which might be considered as a component of metabolic syndrome, varies in severity, ranging from simple hepatic steatosis to non-alcoholic steatohepatitis (NASH), a more progressive form characterized by inflammation, hepatocyte apoptosis and often fibrosis. Since patients with NASH present a higher risk of progressing to cirrhosis, hepatocellular carcinoma and ultimately, liver-related mortality² than patients with steatosis, it is important to understand the mechanisms underlying this transition.

The endoplasmic reticulum (ER) plays a vital role in maintaining cellular and organism metabolic homeostasis. Upon obesity-associated metabolic perturbation, the ER triggers an evolutionarily conserved unfolded protein response (UPR) to reestablish normal ER homeostasis³. The individual branches of the UPR consist of three transmembrane sensors: inositol-requiring enzyme 1 (IRE1α), protein kinase RNA-like ER kinase (PERK) and activating transcription factor 6 (ATF6), which all bind intraluminally to the chaperone GRP78 in unstressed conditions. Prolonged ER stress shifts the UPR from an adaptive to a pro-apoptotic response mediated by C/EBP homologous protein (CHOP)⁴. An exacerbated ER stress response reported in obese mice^{5,6} and humans^{7,8} has been found to play key role in diabetes and NAFLD⁹⁻¹¹.

The NLRP3 inflammasome ignites inflammation and insulin resistance in obesity via the caspase-1- or -11-dependent proteolytic maturation of the proinflammatory cytokines interleukin (IL)-1β and IL-18¹². Persistent NLRP3 inflammasome activation also triggers pyroptosis, a form of programmed cell death. Pyroptotic hepatocyte death, as a consequence of global-specific NLRP3 inflammasome activation, contributes to NASH progression^{13,14}. We recently showed that ER stress activates the NLRP3 inflammasome and hepatocyte death in liver disorders¹⁵.

With the global epidemic of obesity, the liver diseases increase in Western countries. Nonalcoholic fatty liver disease (NAFLD) is a major cause of liver disease worldwide. There's currently no specific medication for NAFLD. Making healthy lifestyle choices can help and treatment may be recommended for associated conditions (high blood pressure, diabetes and cholesterol) or complications. Thus, there is a need to understand the liver disease and find innovative therapeutic strategy to treat liver diseases notably NAFLD.

### SUMMARY OF THE INVENTION

The invention relates to a method for treating a subject suffering from a liver disease comprising a step of administering said subject with a therapeutically effective amount of an inhibitor of the endoribonuclease activity of IRE1α. In particular, the invention is defined by claims.

### DETAILED DESCRIPTION OF THE INVENTION

Inventors have shown that in livers of tunicamycin-treated BI-1−/− mice aIRE1α-dependent NLRP3 inflammasome activation, a hepatocyte death, a fibrosis and a dysregulated lipid homeostasis that led to liver failure within a week. They also analysed human NAFLD liver biopsies which revealed that BI-1 downregulation parallel to the upregulation of IRE1′α RNase signalling. To test whether the pharmacological inhibition of IRE1α endoribonuclease activity would block the transition to NASH, mice were injected with the small molecule STF-083010 twice a week for 2 weeks towards the end of a 3-month HFD. In BI-1−/− mice, STF-083010 treatment effectively counteracted IRE1a endoribonuclease activity, improving glucose tolerance and rescuing from NASH. The hepatocyte-specific role of IRE1α's RNase activity in mediating NLRP3 inflammasome activation and programmed cell death was confirmed in primary mouse hepatocytes through knockdown experiments and with STF-083010. Thus, targeting IRE1α-dependent NLRP3 inflammasomesignaling with pharmacological agents or via BI-1 may represent a tangible therapeutic target for NASH.

Accordingly, the present invention relates to a method for treating a subject suffering from a liver disease comprising a step of administering said subject with a therapeutically effective amount of an inhibitor of the endoribonuclease activity of IRE1α.

As used herein, the terms “treating” or “treatment” refer to both prophylactic or preventive treatment as well as curative or disease modifying treatment, including treatment of subject at risk of contracting the disease or suspected to have contracted the disease as well as subject who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a subject during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a subject during treatment of an illness, e.g., to keep the subject in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., pain, disease manifestation, etc.]).

As used herein, the term “liver disease” refers to any disturbance of liver function that causes illness. The liver is responsible for many critical functions from protein production and blood clotting to cholesterol, glucose (sugar), and iron metabolism. When it becomes diseased or injured, the loss of those functions can cause significant damage to the body. Liver disease is also referred to as hepatic disease. The liver disease is selected from the group consisting of: Nonalcoholic fatty liver disease (NAFLD), Non-alcoholic steatohepatitis (NASH), fibrotic NASH or liver cancer. In the context of the invention, the liver disease is NAFLD. As used herein, the term “NAFLD” refers to nonalcoholic fatty liver disease. NAFLD occurs when the fat is deposited in the liver due to other causes than alcohol excessive consumption, typically caused by drugs, soft drinks. NAFLD can cause liver injury and metabolic changes that can lead to liver cancer. The spectrum of NAFLD is wide ranging from hepatic steatosis to nonalcoholic steatohepatitis (NASH), cirrhosis and liver cancer (hepatocellular carcinoma).

As used herein, the term “hepatic steatosis” refers to a benign non-progressive clinical stage. Hepatic steatosis is characterized by triglyceride accumulation in hepatocytes. As used herein, the term “NASH” refers to a progressive form and advanced form of non-alcoholic fatty liver disease (NAFLD) combining steatosis, hepatocyte death and inflammation. When the buildup of fat causes inflammation and damage, it is known as NASH, which can lead to scarring of the liver. Scarring of the liver is a potentially life-threatening condition called cirrhosis. As used herein, the term “fibrotic NASH” refers to a more harmful stage combining fibrosis in addition to steatosis, inflammation and hepatocyte death.

As used herein, the term “liver cancer” also known as hepatic cancer and primary hepatic cancer refers to the cancer which starts in liver when cells begin to grow out of control. Liver cancer is known to develop in patients with NASH with or without cirrhosis. Cirrhosis is a complication of liver disease which involves loss of liver cells and irreversible scarring of the liver. There are many causes of cirrhosis including chemicals (such as alcohol, fat, and certain medications), viruses, toxic metals (such as iron and copper that accumulate in the liver as a result of genetic diseases), and autoimmune liver disease.

In a particular embodiment, the liver cancer is hemangioma. Hemangioma is a benign liver tumor and starts in blood vessels. Most hemangiomas of the liver cause no symptoms and do not need treatment.

In another embodiment, the liver cancer is hepatic adenoma. It is a benign tumor that starts from hepatocytes (main type of liver cells).

In a particular embodiment, the liver cancer is focal nodular hyperplasia (FNH). FNH is a tumor-like growth made up of several cell types (hepatocytes, bile duct cells, and connective tissue cells).

In another embodiment, the liver cancer is hepatocellular carcinoma (also called as hepatocellular cancer, HCC). This type of liver cancer is the most common form of liver cancer in adults. It has different growth patterns: 1) some begin as a single tumor that grows larger; only late in the disease does it spread to other parts of the liver, and 2) a second type seems to start as many small cancer nodules throughout the liver, not just a single tumor.

As used herein, the term “subject” refers to any mammals, such as a rodent, a feline, a canine, and a primate. Particularly, in the present invention, the subject is a human afflicted with or susceptible to be afflicted with at least one of liver disease as described above. More particularly, the subject is afflicted with or susceptible to be afflicted with NAFLD, NASH or hepatocellular carcinoma.

As used herein, the term “IRE1α” refers to inositol-requiring enzyme 1 (IRE1) and in humans is encoded by the ERN1 gene. It is an endoplasmic reticulum (ER)-resident transmembrane signaling protein and a cellular stress sensor. This protein harbors a cytosolic dual kinase/endoribonuclease activity required for adaptive responses to micro-environmental changes. In the context of the invention, the inventors have shown that blocking IRE1α RNase activity in HFD-fed mice limits lipogenesis priming, hyperglycemia and collagen accumulation in livers of BI-1-deficient mice and does not affect liver appearance in ND-fed mice. Moreover, they have shown that targeting IRE1α RNase activity protects against terminal UPR-driven metabolic dysfunction, sterile inflammation, cell death and fibrosis in the liver. Thus, they propose to target IRE1α RNase activity to treat liver dieses such as NAFLD or liver cancer.

As used herein, the term “RNase activity of IRE1” refers to the activity of the endoribonuclease domain of IRE1 which degrades specific RNA (mRNA or microRNA) to avoid their translation or their cellular activity, an activity known as the RIDD (regulated IRE1-dependent decay of RNA), or contributes to the splicing XBP1 (X-box-binding protein 1) mRNA to change the reading frame leading to the production of a novel protein (XBP1s), a potent unfolded-protein response transcriptional activator. Typically, the IRE1 endonuclease activity is based on recognition of an RNA stem loop structure found twice in substrates HAC1 mRNA in yeast or XBP1 mRNA in metazoans. Cleavage of HAC1 or XBP1 mRNA occurs at both sites resulting in an mRNA fragment whose two ends are ligated in a unique splicing event (Ron et al.; Lee et al.). The spliced HAC1 or XBP1 mRNAs encode transcription factor that activate numerous target genes, including genes involved in the unfolded protein response (UPR). The UPR is a signal transduction cascade that occurs in response to the accumulation of misfolded proteins in the ER.

As used herein, the term “inhibitor of endoribonuclease activity of IRE1α” has its general meaning in the art and refers to any compound, natural or synthetic, that blocks, suppresses, or reduces (including significantly) the expression or activity of IRE1α. More particularly, in the context of the invention, the inhibitor is an inhibitor of RNase activity of IRE1α. Typically, such inhibitor inhibits the degradation of specific RNA (mRNA or microRNA) performed by IRE1α RNase.

In a particular embodiment, the inhibitor of endoribonuclease activity of IRE1α is a peptide, peptidomimetic, small organic molecule, aptamers or single domain antibody.

In some embodiments, the inhibitor of endoribonuclease activity of IRE1α is a polypeptide or fragment thereof. As used herein, the term “polypeptide” refers both short peptides with a length of at least two amino acid residues and at most 10 amino acid residues, oligopeptides (11-100 amino acid residues), and longer peptides (the usual interpretation of “polypeptide”, i.e. more than 100 amino acid residues in length) as well as proteins (the functional entity comprising at least one peptide, oligopeptide, or polypeptide which may be chemically modified by being glycosylated, by being lipidated, or by comprising prosthetic groups). In another embodiment, the peptide competes with IRE1α. In another embodiment, the peptide competes with a substrate of IRE1α.

The polypeptides of the invention may be produced by any suitable means, as will be apparent to those of skill in the art. In order to produce sufficient amounts of polypeptides for use in accordance with the present invention, expression may conveniently be achieved by culturing under appropriate conditions recombinant host cells containing the polypeptide of the invention. In particular, the polypeptide is produced by recombinant means, by expression from an encoding nucleic acid molecule. Systems for cloning and expression of a polypeptide in a variety of different host cells are well known. When expressed in recombinant form, the polypeptide is in particular generated by expression from an encoding nucleic acid in a host cell. Any host cell may be used, depending upon the individual requirements of a particular system. Suitable host cells include bacteria mammalian cells, plant cells, yeast and baculovirus systems. Mammalian cell lines available in the art for expression of a heterologous polypeptide include Chinese hamster ovary cells. HeLa cells, baby hamster kidney cells and many others. Bacteria are also preferred hosts for the production of recombinant protein, due to the ease with which bacteria may be manipulated and grown. A common, preferred bacterial host is E. coli. The polypeptides of the invention and fragments thereof according to the invention can exhibit post-translational modifications, including, but not limited to glycosylations, (e.g., N-linked or O-linked glycosylations), myristylations, palmitylations, acetylations and phosphorylations (e.g., serine/threonine or tyrosine). In some embodiments, it is contemplated that polypeptides used in the therapeutic methods of the present invention may be modified in order to improve their therapeutic efficacy. Such modification of therapeutic compounds may be used to decrease toxicity, increase circulatory time, or modify biodistribution. For example, the toxicity of potentially important therapeutic compounds can be decreased significantly by combination with a variety of drug carrier vehicles that modify biodistribution. In example adding dipeptides can improve the penetration of a circulating agent in the eye through the blood retinal barrier by using endogenous transporters.

In some embodiments, the inhibitor of endoribonuclease activity of IRE1α is a peptidomimetic. As used herein, the term “peptidomimetic” means a peptide-like molecule that has the activity of the peptide upon which it is structurally based. Such peptidomimetics include chemically modified peptides, peptide-like molecules containing non-naturally occurring amino acids, and peptoids, and have an activity such as selective homing activity of the peptide upon which the peptidomimetic is derived (see, for example, Goodman and Ro, Peptidomimetics for Drug Design, in “Burger's Medicinal Chemistry and Drug Discovery” Vol. 1 (ed. M. E. Wolff; John Wiley & Sons 1995), pages 803-861). Peptidomimetics may be designed in order to increase peptide stability, bioavailability, solubility, etc.

In a particular embodiment, the inhibitor of endoribonuclease activity of IRE1α is an aptamer. Aptamers are a class of molecule that represents an alternative to antibodies in term of molecular recognition. Aptamers are oligonucleotide or oligopeptide sequences with the capacity to recognize virtually any class of target molecules with high affinity and specificity.

In a particular embodiment, the inhibitor of endoribonuclease activity of IRE1α is a small organic molecule. As used herein, the term “small organic molecule” refers to a molecule of a size comparable to those organic molecules generally used in pharmaceuticals. The term excludes biological macromolecules (e.g., proteins, nucleic acids, etc.). Preferred small organic molecules range in size up to about 5000 Da, more preferably up to 2000 Da, and most preferably up to about 1000 Da.

In a particular embodiment, the inhibitor of endoribonuclease activity of IRE1α is an RNase domain inhibitor.

In a particular embodiment, the inhibitor of endoribonuclease activity of IRE1α is STF083010. As used herein, the term “STF083010” has its general meaning in the art and refers to N-[(2-Hydroxy-1-naphthalenyl)methylene]-2-thiophenesulfonamide. The Cas number of this molecule is: 307543-71-1.

In a particular embodiment, the inhibitor of endoribonuclease activity of IRE1α is salicylaldehyde and its analogs, as described in Volkmann et al 2011, JBC.

In a particular embodiment, the inhibitor of endoribonuclease activity of IRE1α is 4μ8c, as described in Cross et al 2012, PNAS. The Cas number of this molecule is 14003-96-4.

In a particular embodiment, the inhibitor of endoribonuclease activity of IRE1α is MKC-3946, as described in Mimura et al 2012, Blood.

In a particular embodiment, the inhibitor of endoribonuclease activity of IRE1α is MKC-8866.

In a particular embodiment, the inhibitor of endoribonuclease activity of IRE1α is Toyocamycin, as described in Tashiro et al 2012, Blood Cancer.

In a particular embodiment, the inhibitor of endoribonuclease activity of IRE1α is a type I kinase inhibitor.

In a particular embodiment, the inhibitor of the endoribonuclease activity of IRE1α is type II kinase inhibitor.

In one embodiment, the inhibitor of the RNase activity of endoribonuclease activity of IRE1α is Irestatin.

In one embodiment, the inhibitor of the RNase activity of endoribonuclease activity of IRE1α is MG132.

In one embodiment, the inhibitor of the RNase activity of endoribonuclease activity of IRE1α is 17-AAG.

In one embodiment, the inhibitor of the RNase activity of endoribonuclease activity of IRE1α is 1-NM-PP1.

In one embodiment, the inhibitor of the RNase activity of endoribonuclease activity of IRE1α is Lactacystin.

In one embodiment, the inhibitor of the RNase activity of endoribonuclease activity of IRE1α is 3-methoxy-6-bromosalicylaldehyde.

In one embodiment, the inhibitor of endoribonuclease activity of IRE1α is sunitinib. It is marketed as Sutent and has the cas number: 341031-54-7.

In one embodiment, the inhibitor of endoribonuclease activity of IRE1α is KIRA6. The Cas number of this molecule is 1589527-65-0.

In one embodiment, the inhibitor of endoribonuclease activity of IRE1α is KIRA8.

In one embodiment, the inhibitor of the endoribonuclease activity of IRE1α is a 4-phenylbutyric acid analogue (Zhang H et al 2013, Br J Pharmacol).

In some embodiments, the inhibitor of endoribonuclease activity of IRE1α is single domain antibody. The term “single domain antibody” (sdAb) or “VHH” refers to the single heavy chain variable domain of antibodies of the type that can be found in Camelid mammals which are naturally devoid of light chains. Such VHH are also called “nanobody®”. According to the invention, sdAb can particularly be llama sdAb.

In one embodiment, the inhibitor of endoribonuclease activity of IRE1α is administered in combination with a classical treatment of a liver disease.

Thus, the invention also refers to a method for treating a subject suffering from a liver disease comprising a step of administering said subject with i) a therapeutically effective amount of an inhibitor of the endoribonuclease activity of IRE1α and ii) classical treatment of a liver disease.

As used herein, the term “classical treatment” refers to any compound, natural or synthetic, used for the treatment of a liver disease.

In a particular embodiment, the classical treatment refers to radiation therapy, antibody therapy or chemotherapy.

According to the invention, compound used for the treatment of a liver disease may be selected in the group consisting in: glitazone agent (such as pioglitazone, rosiglitazone, lobeglitazone), vitamin E, statins, synbiotic, steroid-based drug, ursodeoxycholic acid, biguanide agent (such as metformine), inhibitor of alpha-glucosidase, immune checkpoint inhibitor, chemotherapeutic agent, radiotherapeutics agent.

As used herein, the term “chemotherapeutic agent” refers to chemical compounds that are effective in inhibiting tumor growth. Examples of chemotherapeutic agents include multkinase inhibitors such as sorafenib and sunitinib, alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaorarnide and trimethylolomelamine; acetogenins (especially bullatacin and bullatacinone); a carnptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CBI-TMI); eleutherobin; pancratistatin; a sarcodictyin; spongistatin; nitrogen mustards such as chlorambucil, chlornaphazine, cholophosphamide, estrarnustine, ifosfamide, mechlorethamine, mechlorethamine oxide hydrochloride, melphalan, novembichin, phenesterine, prednimus tine, trofosfamide, uracil mustard; nitrosureas such as carmustine, chlorozotocin, fotemustine, lomustine, nimustine, ranimustine; antibiotics such as the enediyne antibiotics (e.g. calicheamicin, especially calicheamicin (11 and calicheamicin 211, see, e.g., Agnew Chem Intl. Ed. Engl. 33: 183-186 (1994); dynemicin, including dynemicin A; an esperamicin; as well as neocarzinostatin chromophore and related chromoprotein enediyne antiobiotic chromomophores), aclacinomysins, actinomycin, authramycin, azaserine, bleomycins, cactinomycin, carabicin, canninomycin, carzinophilin, chromomycins, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idanrbicin, marcellomycin, mitomycins, mycophenolic acid, nogalarnycin, olivomycins, peplomycin, potfiromycin, puromycin, quelamycin, rodorubicin, streptomgrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophospharnide glycoside; aminolevulinic acid; amsacrine; bestrabucil; bisantrene; edatraxate; defo famine; demecolcine; diaziquone; elfornithine; elliptinium acetate; an epothilone; etoglucid; gallium nitrate; hydroxyurea; lentinan; lonidamine; maytansinoids such as maytansine and ansamitocins; mitoguazone; mitoxantrone; mopidamol; nitracrine; pento statin; phenamet; pirarubicin; podophyllinic acid; 2-ethylhydrazide; procarbazine; PSK®; razoxane; rhizoxin; sizofiran; spirogennanium; tenuazonic acid; triaziquone; 2,2′,2″-trichlorotriethylarnine; trichothecenes (especially T-2 toxin, verracurin A, roridinA and anguidine); urethan; vindesine; dacarbazine; mannomustine; mitobromtol; mitolactol; pipobroman; gacytosine; arabinoside (“Ara-C”); cyclophosphamide; thiotepa; taxoids, e.g. paclitaxel (TAXOL®, Bristol-Myers Squibb Oncology, Princeton, N.].) and doxetaxel (TAXOTERE®, Rhone-Poulenc Rorer, Antony, France); chlorambucil; gemcitabine; 6-thioguanine; mercaptopurine; methotrexate; platinum analogs such as cisp latin and carbop latin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RFS 2000; difluoromethylornithine (DMFO); retinoic acid; capecitabine; and pharmaceutically acceptable salts, acids or derivatives of any of the above. Also included in this definition are antihormonal agents that act to regulate or inhibit honnone action on tumors such as anti-estrogens including for example tamoxifen, raloxifene, aromatase inhibiting 4(5)-imidazoles, 4-hydroxytamoxifen, trioxifene, keoxifene, LY 117018, onapristone, and toremifene (Fareston); and anti-androgens such as flutamide, nilutamide, bicalutamide, leuprolide, and goserelin; and pharmaceutically acceptable salts, acids or derivatives of any of the above.

As used herein, the term “radiation therapy” has its general meaning in the art and refers the treatment of cancer with ionizing radiation. Ionizing radiation deposits energy that injures or destroys cells in the area being treated (the target tissue) by damaging their genetic material, making it impossible for these cells to continue to grow. One type of radiation therapy commonly used involves photons, e.g. X-rays. Depending on the amount of energy they possess, the rays can be used to destroy cancer cells on the surface of or deeper in the body. The higher the energy of the x-ray beam, the deeper the x-rays can go into the target tissue. Linear accelerators and betatrons produce x-rays of increasingly greater energy. The use of machines to focus radiation (such as x-rays) on a cancer site is called external beam radiation therapy. Gamma rays are another form of photons used in radiation therapy. Gamma rays are produced spontaneously as certain elements (such as radium, uranium, and cobalt 60) release radiation as they decompose, or decay. In some embodiments, the radiation therapy is external radiation therapy. Examples of external radiation therapy include, but are not limited to, conventional external beam radiation therapy; three-dimensional conformal radiation therapy (3D-CRT), which delivers shaped beams to closely fit the shape of a tumor from different directions; intensity modulated radiation therapy (IMRT), e.g., helical tomotherapy, which shapes the radiation beams to closely fit the shape of a tumor and also alters the radiation dose according to the shape of the tumor; conformal proton beam radiation therapy; image-guided radiation therapy (IGRT), which combines scanning and radiation technologies to provide real time images of a tumor to guide the radiation treatment; intraoperative radiation therapy (IORT), which delivers radiation directly to a tumor during surgery; stereotactic radiosurgery, which delivers a large, precise radiation dose to a small tumor area in a single session; hyperfractionated radiation therapy, e.g., continuous hyperfractionated accelerated radiation therapy (CHART), in which more than one treatment (fraction) of radiation therapy are given to a subject per day; and hypofractionated radiation therapy, in which larger doses of radiation therapy per fraction is given but fewer fractions.

As used herein, the term “immune checkpoint inhibitor” refers to molecules that totally or partially reduce, inhibit, interfere with or modulate one or more immune checkpoint proteins.

As used herein, the term “immune checkpoint protein” has its general meaning in the art and refers to a molecule that is expressed by T cells in that either turn up a signal (stimulatory checkpoint molecules) or turn down a signal (inhibitory checkpoint molecules).

Examples of stimulatory checkpoint include CD27 CD28 CD40, CD122, CD137, OX40, GITR, and ICOS. Examples of inhibitory checkpoint molecules include A2AR, B7-H3, B7-H4, BTLA, CTLA-4, CD277, IDO, KIR, PD-1, PD-L1, LAG-3, TIM-3 and VISTA.

In one embodiment, the inhibitor of endoribonuclease activity of IRE1α is administered in combination with sorafenib.

In a particular embodiment, the method according to the invention, wherein, i) the inhibitor of endoribonuclease activity of IRE1α and ii) classical treatment are used as combined preparation for treating the subject identified as responder to an immune checkpoint inhibitor treatment.

Accordingly, the invention relates to i) an inhibitor of endoribonuclease activity of IRE1α and iii) a classical treatment used as a combined preparation for treating a subject suffering from a liver disease.

In a particular embodiment, i) an inhibitor of endoribonuclease activity of IRE1α and ii) a classical treatment as a combined preparation according to the invention for simultaneous, separate or sequential use for treating a subject suffering from a liver disease.

As used herein, the terms “combined treatment”, “combined therapy” or “therapy combination” refer to a treatment that uses more than one medication. The combined therapy may be dual therapy or bi-therapy.

As used herein, the term “administration simultaneously” refers to administration of 2 active ingredients by the same route and at the same time or at substantially the same time. The term “administration separately” refers to an administration of 2 active ingredients at the same time or at substantially the same time by different routes. The term “administration sequentially” refers to an administration of 2 active ingredients at different times, the administration route being identical or different.

As used herein the terms “administering” or “administration” refer to the act of injecting or otherwise physically delivering a substance as it exists outside the body (e.g., an inhibitor of IRE1α) into the subject, such as by mucosal, intradermal, intravenous, subcutaneous, intramuscular delivery and/or any other method of physical delivery described herein or known in the art. When a disease, or a symptom thereof, is being treated, administration of the substance typically occurs after the onset of the disease or symptoms thereof. When a disease or symptoms thereof, are being prevented, administration of the substance typically occurs before the onset of the disease or symptoms thereof.

A “therapeutically effective amount” is intended for a minimal amount of active agent which is necessary to impart therapeutic benefit to a subject. For example, a “therapeutically effective amount” to a subject is such an amount which induces, ameliorates or otherwise causes an improvement in the pathological symptoms, disease progression or physiological conditions associated with or resistance to succumbing to a disorder. It will be understood that the total daily usage of the compounds of the present invention will be decided by the attending physician within the scope of sound medical judgment. The specific therapeutically effective dose level for any particular subject will depend upon a variety of factors including the disorder being treated and the severity of the disorder; activity of the specific compound employed; the specific composition employed, the age, body weight, general health, sex and diet of the subject; the time of administration, route of administration, and rate of excretion of the specific compound employed; the duration of the treatment; drugs used in combination or coincidential with the specific compound employed; and like factors well known in the medical arts. For example, it is well within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Typically, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the subject to be treated. A medicament typically contains from about 0.01 mg to about 500 mg of the active ingredient, preferably from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

The inhibitors of IRE1α as described above may be combined with pharmaceutically acceptable excipients, and optionally sustained-release matrices, such as biodegradable polymers, to form pharmaceutical compositions. “Pharmaceutically” or “pharmaceutically acceptable” refer to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type. The pharmaceutical compositions of the present invention for oral, sublingual, subcutaneous, intramuscular, intravenous, transdermal, local or rectal administration, the active principle, alone or in combination with another active principle, can be administered in a unit administration form, as a mixture with conventional pharmaceutical supports, to animals and human beings. Suitable unit administration forms comprise oral-mute forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms. Typically, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions. The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. Solutions comprising compounds of the invention as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms. The polypeptide (or nucleic acid encoding thereof) can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin. Sterile injectable solutions are prepared by incorporating the active polypeptides in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion. Some variation in dosage will necessarily occur depending on the condition of the subject being treated. The person responsible for administration will, in any event, determine the appropriate dose for the individual subject.

### Method De Screening

Tests for determining the capacity of a compound to be inhibitor of endoribonuclease activity of IRE1α are well known to the person skilled in the art. Inhibition of the IRE1α may be determined by any techniques well known in the art. For instance, an inhibitor of RNase activity of IRE1 can be identified by carrying out the following steps: i) providing a plurality of test substances ii) determining whether the test substances are inhibitors of RNase activity of IRE1 and iii) positively selecting the test substances that are inhibitor of RNase activity of IRE1. The test substances that have been positively selected may be subjected to further selection steps in view of further assaying its properties for the treatment of cancer. For example, the candidate compounds that have been positively selected may be subjected to further selection steps in view of further assaying its properties on animal models.

The assays may be performed using high throughput screening techniques for identifying test substances for developing drugs (inhibitor of IRE1 RNase activity) that may be useful to the treatment of cancer. High throughput screening techniques may be carried out using multi-well plates (e.g., 96-, 389-, or 1536-well plates), in order to carry out multiple assays using an automated robotic system. Thus, large libraries of test substances may be assayed in a highly efficient manner. Compounds in the library will be applied one at a time in an automated fashion to the wells of the microtitre dishes. Once the test substances which inhbits the RNase activity of IRE1 are identified, they can be positively selected for further characterization. Because this assay can readily be performed in a microtitre plate format, the assays described can be performed by an automated robotic system, allowing for testing of large numbers of test samples within a reasonably short time frame. The assays can be used as a screen to assess the activity of a previously untested compound or extract, in which case a single concentration is tested and compared to controls. These assays can also be used to assess the relative potency of a compound by testing a range of concentrations, in a range of 100 μM to 1 μM, for example, and computing the more efficient concentration.

The invention will be further illustrated by the following figures and examples. However, these examples and figures should not be interpreted in any way as limiting the scope of the present invention.

### FIGURES

FIG. 1A-C: Early Into HFD feeding, limiting the ER stress response by inhibiting IRE1α RNase activity protects from steatosis and hyperglycemia. Starting 2.5 months after HFD feeding, BI-1+/+ and BI-1−/− mice were treated with STF-083010 (30 mg/kg) or vehicle (NT, Kolliphor 16%) twice a week for 2 weeks before sacrifice. (A) Relative liver triglyceride levels. Analysis of ER stress markers by (B) immunoblot with respect to the loading control HSP90, and (C) qPCR, genes significantly (#p<0.05) different in expression comparing NT HFD-fed BI-1+/+ and BI-1−/− mice. n=6; $p<0.05 when comparing treated to untreated counterpart.

FIG. 2A-C: Blocking IRE1α RNase activity in HFD-fed mice limits lipogenesis priming, hyperglycemia and collagen accumulation in livers of BI-1-deficient mice and does not affect liver appearance in ND-fed mice. (A) Protocol timeline for vehicle (NT, Kolliphor 16%) or STF-083010 (30 mg/kg) injections in HFD-fed BI-1+/+ and BI-1−/− mice. (B) qPCR analysis of hepatic genes involved in lipid synthesis and metabolism (n=3). Genes are significantly (#p<0.05) different in expression comparing NT HFD-fed BI-1+/+ and BI-1−/− mice. $p<0.05 when comparing treated to untreated counterpart. (C) Blood glucose concentrations in fed BI-1+/+ and BI-1−/− mice (n=6).

FIG. 3A-B: Inhibition of IRE1α RNase signalling with STF potentiates the effectiveness of Sorafenib in reducing liver cancer cell proliferation and inducing apoptosis. Effect of Sorafenib (SORA, 5 μM), STF-083010 (STF, 60 μM) and their combination on (A) liver cancer cell proliferation (HepG2, Huh7 and Hep3B) and on (B) inducing apoptosis. STF-083010 was added one hour before SORA. Stimulations were performed during 24 hours. $p<0.05 when comparing treated to untreated counterpart. Results are expressed as mean with SEM *p<0.05, **p<0.01, ***p<0.001 and ****p<0.0001.

FIG. 4: Hyperactive IRE1α RNase triggers secretion of pro-inflammatory markers. Effect of Sorafenib (SORA, 51M), Ac-YVAD-CMK (YVAD, 25 μM) and their combination on liver cancer cell proliferation (HepG2) and on inducing apoptosis. Ac-YVAD-CMK (YVAD) was added one hour before SORA. Stimulations were performed during 24 hours. $p<0.05 when comparing treated to untreated counterpart. Results are expressed as mean with SEM *p<0.05, **p<0.01, ***p<0.001 and ****p<0.0001.

FIG. 5A-B: Co-treatment enhancing apoptosis via IRE1α kinase activity. Effect of Sorafenib (SORA, 5 μM), STF-083010 (STF, 60 μM) and their combination on HepG2 cells proliferation with or without (A) JNK inhibitor SP600125 (SP, 50 μM) pre-treatment or (B) Necrostatin (Nec-1, 50 μM) pre-treatment. Stimulations were performed during 24 hours. $p<0.05 when comparing treated to untreated counterpart. Results are expressed as mean with SEM *p<0.05, **p<0.01, ***p<0.001 and ****p<0.0001.

FIG. 6A-B: Inhibition of IRE1α RNase activity with STF potentiates Sorafenib action in vivo. (A) Protocol timeline for Sorafenib (30 mg/kg i.p. daily) and/or STF-083010 (30 mg/kg i.p. twice a week) injections in mice with xenograft HepG2 cells. (B) Effect of SORA, STF, and their combination on tumor volume in mice. $p<0.05 when comparing treated to untreated counterpart. Results are expressed as mean with SEM *p<0.05.

### EXAMPLE

### Material & Methods

### Animal Care and Experimentation

All animal procedures were conducted in compliance with French guidelines for the humane care and use of experimental animals. Male mice with targeted disruption of the BI-1 gene representing BI-1+/+(WT) and BI-1−/− littermates on a C57BL/6 background were obtained from Dr. John C. Reed at the Stanford-Burnham Institute for Medical Research (La Jolla, CA, USA)17,18. Experiments conducted with 12-week old mice included: 1) Mice injected intraperitoneally with TM (1 mg/kg) or vehicle and sacrificed 48 hr after treatment (unless otherwise indicated), 2) Animals either fed a ND (A04—Safe Diet, Augy, France) or HFD (60 kJ % fat, D12492-ssniff, Soest, Germany) for up to 9 months, 3) Towards the end of a 3-month ND or HFD, mice were injected with STF-083010 (30 mg/kg) or vehicle twice a week for 2 weeks before sacrifice. Mice were housed in a controlled environment with 12 hr light/dark cycles with water available ad libitum.

Histological Evaluation

Liver tissue specimens were fixed in 10% buffered formalin, embedded in paraffin, sectioned (5 μm thick) and stained with either H&E, Masson's trichrome, MPO or TUNEL (Roche Molecular Biochemicals, Meylan, France). Specimens were evaluated by microscopy in a blinded manner by a liver pathologist.

Electron Microscopy

Mouse livers were dissected, immerged in fixative and processed as described in Supporting Methods. Contrasted ultrathin sections (70 nm) were analyzed under a JEOL 1400 transmission electron microscope equipped with a Morada Olympus CCD camera. IMOD software was used to analyze images and delineate major cellular structures.

Real-Time Quantitative PCR Analysis

Total RNA was extracted from liver tissue or cells then reverse-transcribed as previously described15. Real-time quantitative PCR was performed using the ABI PRISM 7500/Step-One Fast Real Time PCR System following the manufacturer's protocols in C3M genomics facilities. See the Supporting Methods for TaqMan gene expression assays.

Immunoblot Analysis

Total protein was isolated from snap-frozen livers or primary hepatocytes homogenized in detergent-containing buffer, normalized for protein content (40 μg/tissue sample and 20 μg/cell sample), and analyzed by SDS-PAGE (8-15% gels) immunoblotting as previously described15. Western blot analyses were performed using antibodies described in Supporting Methods and detected with an enhanced chemiluminescence method (Amersham Biosciences, Piscataway, NJ, USA). Immunoblots were scanned and the signals were quantified using ImageJ software.

Biochemical Analysis and Cytokine Measurement

Serum AST and ALT levels were determined using a standardized UV test after activation with pyridoxal-phosphate and serum triglyceride levels were determined by standardized enzymatic colorimetric assay (Roche-Hitachi analyzer Cobas 8000, Meylan, France). Hepatic triglyceride content was measured using a Triglycerides FS 10′ kit (DiaSys, Holzheim, Germany). Cytokines were quantitatively measured by flow cytometry as described15.

Cellular Models, Treatments and Viability Assay

Hepatocytes from mouse liver were isolated by a two-step collagenase procedure, as we described18,23 and cultured in media detailed in Supporting Methods. When indicated, primary hepatocytes were transfected with Stealth siRNA (targeting BI-1, IRE1α, XBP1 or CHOP with the corresponding control of Low or Medium CG) at 30 nM using Lipofectamine RNAiMAX (ThermoFisher Scientific, Courtaboeuf, France) according to the manufacturer's instructions. After 48 hr of transfection, cells were treated with TM (1 μg/ml) or LPS (100 ng/ml) for the determined times. Primary hepatocytes isolated from the livers of BI-1−/− and WT mice were treated with TM or LPS in the presence or absence of STF-083010 (60 μM) or Ac-YVAD-CMK (25 μM). Cell viability was determined by a colorimetric assay (MTT) detailed in Supporting Methods. Results are presented as a percentage of the control values.

Human Studies

Morbidly obese patients: 10 patients were recruited through the Department of Digestive Surgery and Liver Transplantation (Nice hospital) where they underwent bariatric surgery for their morbid obesity. Bariatric surgery was indicated for these patients in accordance with French guidelines. The characteristics of the study groups for the gene expression are described in Table 1. Before surgery, fasting blood samples were obtained and used to measure alanine and aspartate transaminases (ALT and AST, respectively), glucose, insulin and HbA1c. Insulin resistance was calculated using the homeostatic model assessment (HOMA-IR) index. Liver biopsies were obtained during surgery and no ischemic preconditioning had been performed. Three histopathological features were semi-quantitatively evaluated: grade of steatosis (0, <5%; 1, 5-30%; 2, >30-60%; 3, >60%), lobular inflammation (0, no inflammatory foci; 1, <2 inflammatory foci per 200× field; 2, 2-4 inflammatory foci per 200× field; 3, >4 inflammatory foci per 200× field, hepatocellular ballooning (0, none; 1, few balloon cells; 2, many cells/prominent ballooning).

Control participants: Liver tissue was obtained from 5 lean participants (5 women; age, 44±9 years; BMI, 21±1.9 kg/m2) undergoing partial hepatectomy for benign tumours (neighbour tissues from four adenoma and one focal nodular hyperplasia). Three participants underwent a left lobectomy or a bisegmentectomy without ischemic preconditioning and two patients underwent a right hepatectomy with a potential ischemic preconditioning (missing data). Liver samples did not display any hepatic steatosis, inflammation or fibrosis. All participants gave their informed written consent to participate in this study in accordance with French legislation regarding Ethics and Human Research (Huriet-Serusclat law). The “Comité Consultatif de Protection des Personnes dans la Recherche Biomédicale de Nice” approved the study (07/04:2003, N 03.017).

Statistics

Data are represented as means ± SEM. Differences in the mean values between 2 groups were assessed using a 2-tailed Student's t test. Differences in the mean values among more 2 groups were assessed by 1-way or 2-way ANOVA and corrected for multiple comparisons with Bonferonni or Sidak post-hoc testing, respectively. All analyses were performed with GraphPad Prism 7 software.

Results

BI-1 deficiency predisposes to unresolved ER stress after an acute challenge leading to hepatic lipid deposition and metabolic collapse.

We investigated the possibility that the loss of BI-1 expression alters the magnitude of the hepatic ER stress response. Forty-eight hours after TM injection, the livers of BI-1^{-/-} turned much paler than WT control organs (data not shown). The majority of TM-treated BI-1^{-/-} mice presented grade-3 steatosis characterized by macrovesicular and microvesicular steatosis, contrasting with TM-treated WT mice that mostly developed grade-1 microvesicular steatosis (data not shown). An almost 3-fold increase in hepatic triglyceride content was observed in BI-1^{-/-} TM mice (data not shown), in accordance with significantly higher liver weight relative to body weight (data not shown). Transmission electron microscopy (TEM) revealed lipid droplets greater in number and size accumulating in hepatocytes after TM injection, especially in BI-1^{-/-} mice (data not shown). The genes encoding for the lipid droplet markers ADFP and CIDEC were significantly upregulated in TM-treated BI-1^{-/-} compared to WT mice, as well as the genes encoding fatty acid transporter proteins (CD36 and FAT)). However, both the secretory apolipoprotein ApoB and the microsomal triglyceride transfer protein (MTTP) involved in very-low-density lipoprotein secretion were downregulated, possibly explaining the massive hepatic build-up of lipid content in BI-1^{-/-} TM mice. While certain key genes involved in lipid and glucose metabolism were slightly upregulated at the basal level in BI-1^{-/-} compared to WT mice, most were significantly downregulated after TM injection in both genotypes, though to a much greater extent in BI-1^{-/-} TM mice (data not shown). In contrast, c-JUN was upregulated in BI-1^{-/-} mice, especially after TM injection. Elevated hepatic expression of c-JUN can result from C/EBPα deletion²⁴. A collapse in C/EBPα, but also G6P and FASN was thus seen in BI-1^{-/-} TM mice. Supporting the concept that ER stress leads to insulin resistance, we observed a decline in the phosphorylation status of the kinase AKT at Ser473 in TM-treated WT mice that was more severe in BI-1^{-/-} TM mice. Thus, the exaggerated ER stress of TM-treated BI-1^{-/-} mice causes liver steatosis and metabolic collapse likely through enhanced fatty acid uptake, inefficient β-oxidation and reduced fatty acid release.

While TEM revealed that TM injection causes swelling and breakdown of the lamellar structure of the ER in hepatocytes, cells from BI-1^{-/-} TM mice featured greater loss of ER structural integrity and more mitochondria-associated ER membranes. The protein levels of sXBP1, CHOP and GRP78 were highest in livers from BI-1^{-/-} TM mice. While hepatic cATF6 expression also increased in BI-1^{-/-} TM mice, ATF4 expression did not change reflecting unperturbed PERK signaling. At 24 hours postinjection, TM markedly reduced BI-1 mRNA levels, opposite increases in XBP1 and CHOP mRNA levels that remained significantly elevated 48 hours post-TM in BI-1^{-/-} compared to WT livers. BI-1 deficiency thus favors enhanced and prolonged ER stress, particularly through the IRE1α branch with persistent CHOP upregulation.

Lack of BI-1 favors NLRP3 inflammasome overactivation leading to hepatic injury after TM challenge.

We next investigated whether BI-1 deficiency would favor activation of the NLRP3 inflammasome. After 48 hours of TM, livers from BI-1^{-/-} mice contained higher caspase-11 levels than WT organs, correlating with an increased abundance of its substrate IL-1β as well as its sensor NLRP3, and elevated levels of thioredoxin-interacting protein (TXNIP). The mRNA levels coding for inflammasome components (caspase-11-11, NLRP3) were significantly upregulated in BI-1^{-/-} livers 24 hours postinjection, and these mRNA levels remained elevated in BI-1^{-/-} mice while they returned to baseline in WT mice 48 hours postinjection. In parallel, we found a TM-induced increase in the mRNA coding for proinflammatory cytokines (IL-1β, TNFα and IL-6), chemokines (MCP1 and CXCL1) and toll-like receptor (TLR4) mRNA in BI-1^{-/-} mice 24 and 48 hours postinjection compared to WT mice. Accordingly, TM injection induced elevated circulating levels of IL-1β, TNFα and IL-6 in BI-1^{-/-} mice. Serum levels of MCP1 and CXCL1 were also highest in TM-treated BI-1^{-/-} mice, suggesting neutrophil recruitment into the liver²⁵ that was confirmed by an increase in myeloperoxidase (MPO)-positive cells associated with lipid-laden hepatocytes, similarly to what was reported in human NASH patients²⁶.

Excess fibrous connective tissue was present in TM-treated BI-1^{-/-} mice, as revealed by Masson's trichrome staining and TEM. Acute ER stress appears to intensify the fibrogenic phenotype of BI-1^{-/-} mice, as further shown by the significant increase in the mRNA levels of the metalloproteinase inhibitor TIMP1. Liver fibrosis has been linked to excessive inflammatory signaling as a consequence of hepatocyte death^{11,27}. Aspartate (AST) and alanine (ALT) transaminases were significantly elevated in BI-1^{-/-} TM sera. Untreated BI-1^{-/-} livers already exhibited an increase in TUNEL-positive hepatocytes over WT controls, and TM injection further aggravated the predisposed phenotype of BI-1^{-/-} mice to apoptosis and/or pyroptosis. Immunoblotting and mRNA analysis revealed a significantly higher ratio of pro-apoptotic BAX to pro-survival BCL2 protein in TM-treated BI-1^{-/-} mice compared to WT controls presumably as a consequence of CHOP upregulation²⁸.

We next explored the capacity of BI-1^{-/-} mice to recover from the normally transient hepatotoxic effects of TM. Mice treated with TM displayed decreased serum glucose levels 24 hours postinjection. Unlike WT mice that recovered after 48 hours and survived the TM challenge, BI-1^{-/-} mice continued to manifest hypoglycemia until they died within 5 days of TM injection. Moreover, both the liver and kidney from TM-treated BI-1^{-/-} mice presented a pale, discolored appearance 72 hours postinjection. These results emphasize of BI-1 importance in protecting the liver from ER stress-induced inflammation, hepatocyte death, fibrosis and liver failure.

In Livers of NAFLD Patients, BI-1 Downregulation is Accompanied by Greater IRE1α RNase Signaling

The relevance of IRE1α signaling in the context of human NAFLD was then investigated in liver biopsies obtained from a small group of morbidly obese patients (Table 1). Hepatic expression of BI-1 was specifically downregulated by 20% in morbidly obese patients with normal liver histology and 36% in obese patients with NAFLD, compared to lean participants. On the contrary, significant increases in XBP1 and CHOP mRNA levels were observed predominantly in obese patients with NAFLD.

BI-1 deficiency aggravates the metabolic impact of chronic ER stress in mice.

The impact of BI-1 deficiency was further investigated in mice fed a long-term HFD to more closely mimic NAFLD installment. BI-1^{-/-} mice were fed a normal diet (ND) or HFD for 9 months, the time previously reported to induce inflammasome activation and steatohepatitis in WT mice²⁹. Histological analysis of liver sections revealed important steatosis and elevated hepatic triglyceride levels in BI-1^{-/-} HFD mice. The mRNA levels of major metabolic markers (PEPCK, DGA72 and PPARα) were significantly upregulated in livers of HFD-fed BI-1^{-/-} mice. HFD caused an undistinguishable increase in body weight in BI-1^{-/-} and WT mice, but significantly higher baseline glucose levels in BI-1^{-/-} mice both in ND and in HFD conditions when compared to WT controls. Investigating signs of diabetes, HFD feeding led to glucose intolerance and a poor insulin response most significantly in BI-1^{-/-} mice. Phospho-AKT and that of its target GSK3, were significantly decreased in BI-1^{-/-} HFD mice.

Concerning ER stress pathways, HFD-induced higher levels of sXBP1 protein in BI-1^{-/-} than in WT livers, reflecting overactive IRE1α RNase, while proteins of the ATF6 and PERK pathways did not significantly differ in expression between genotypes. Lower mRNA levels of hepatic BI-1 opposed higher XBP1 and CHOP/GRP78 ratio levels significantly in BI-1^{-/-} HFD mice compared to WT mice. Hence, a deficiency in BI-1, resulting in unrestrained IRE1α-XBP1 signaling, may predispose to metabolic risk factors associated with the development of type-2 diabetes.

Long-term HFD overwhelms the NLRP3 inflammasome and provokes NASH in BI-1-deficient mice.

We reasoned that the absence of BI-1 might aggravate the vulnerability towards HFD-induced NLRP3 inflammasome activation, thus promoting inflammation and programmed cell death. HFD caused active caspase-1/-11 and IL-1β accumulation in livers of BI-1^{-/-} mice. Hepatic mRNA levels of caspase-1 NLRP3, and ASC were significantly upregulated in BI-1^{-/-} HFD compared to WT mice. Similarly, IL-1β, TNFα, IL-6, MCP1 and TLR4 were significantly increased in HFD-fed BI-1^{-/-} mice. Liver histology analysis revealed the presence of inflammatory foci in HFD-fed mice, though more so in BI-1^{-/-} compared to WT mice (data not shown). We found that BI-1^{-/-} mice fed a HFD for 9 months presented increased collagen deposition in their livers. Such livers expressed high mRNA levels of fibrosis markers including TIMP1, Col6a3 and aSMA. BI-1^{-/-} HFD mice presented a greater number of TUNEL-positive hepatocytes than WT HFD mice, correlating with an enhanced BAX/BCL2 ratio at the protein and mRNA levels. In summary, BI-1 deficiency favors an aggravation of HFD-induced inflammasome activation and inflammation, hepatocyte death and consequent liver fibrosis.

Hepatocytes depleted of BI-1 show metabolic disruption due to hyperactivated IRE1α RNase signaling.

To determine the origin of the BI-1 contribution, we performed fractionation of WT liver into hepatic parenchymal versus non-parenchymal cells. BI-1 mRNA levels were significantly higher in hepatocytes compared to the non-parenchymal fraction of the liver We then explored the metabolic consequences of BI-1 invalidation in primary hepatocytes that exhibited more vesicles that stain with the lipophilic dye Oil Red O than control cells. Accordingly, primary hepatocytes lacking BI-1 expression presented significantly higher mRNA levels of major enzymes involved in lipogenesis. BI-1-silenced hepatocytes were predisposed to activated IRE1α signaling, as seen by the upregulation of XBP1 and CHOP and of target proteins especially 24 hours after addition of TM or bacterial lipopolysaccharide (LPS). Compared to control hepatocytes, BI-1-silenced cells also displayed significantly higher proinflammatory caspase mRNA levels and higher caspase-1, NLRP3 and TXNIP protein levels when treated with TM or LPS. BI-1 might protect against IRE1α-dependent NLRP3 inflammasome activation in primary hepatocytes through CHOP and/or TXNIP activation.

We explored potential mechanisms that may underlie the disproportionate ER stress responses in BI-1^{-/-} hepatocytes by knocking down each member of the IRE1α-XBP1-CHOP axis. While depletion of IRE1α lowered the mRNA levels of XBP1 and CHOP in primary hepatocytes isolated from BI-1^{-/-} and WT mice, it did not limit caspase-1 or NLRP3 hepatocyte mRNA expression. Silencing CHOP did not affect IRE1α or XBP1 mRNA levels in BI-1^{-/-} and WT primary hepatocytes, but significantly lowered caspase-1 and NLRP3 in primary WT (but not BI-1^{-/-}) hepatocytes. Remarkably, silencing XBP1 led to a significant decrease in the mRNA levels coding for ER stress markers and NLRP3 inflammasome components in WT and more so in BI-1^{-/-} hepatocytes. Accordingly, the knockdown of XBP1 prevented the accumulation of p-IRE1α, CHOP, caspase-1, NLRP3 and TXNIP in primary hepatocytes isolated from BI-1^{-/-} mice. This suggests that the IRE1α RNase function of activating XBP1 contributes to the BI-1^{-/-} hepatocyte phenotype.

Inhibition of IRE1α RNase signaling corrects the phenotype of primary BI-1^{-/-} hepatocytes.

Treatment of primary hepatocytes from BI-1^{-/-} or WT mice with STF-083010, a specific inhibitor of IRE1α RNase activity^{30,31} caused a decrease in XBP1 mRNA levels and sXBP1 protein in BI-1^{-/-} cells. The overexpression of active caspase-11 and NLRP3 of LPS-treated BI-1^{-/-} primary hepatocytes was also limited after STF-083010 treatment. Primary BI-1^{-/-} hepatocytes were more sensitive to TM and LPS-induced cell death than WT cells. When STF-083010 was co-treated with TM and LPS, it improved the viability of BI-1^{-/-} hepatocytes. However, STF-083010 alone compromised the viability of WT hepatocytes, perhaps because such cells need some baseline level of the UPR to maintain homeostasis. Cell death caused by TM or LPS in BI-1^{-/-} hepatocytes was also inhibited by Ac-YVAD-CMK, a caspase-1/-11 inhibitor, however not more efficiently than STF-083010. These results suggest that IRE1α RNase inhibition by STF-083010 may reverse the phenotype of BI-1^{-/-} hepatocytes. We performed complementary experiments to question whether a hyperactive NLRP3 inflammasome could impact the properties of macrophages and found that BI-1 deficiency did not alter the macrophage colony-stimulating factor-mediated macrophage differentiation of bone marrow monocytes (data not shown). In addition, as reported by Tufanli et al., we confirmed that IRE1α, through sXBP1, regulates IL-1β secretion in LPS and lipid-stressed bone marrow-derived macrophages; however, BI-1 deficiency did not alter the sensitivity to such stimulations (data not shown)³².

Inhibition of IRE1α RNase activity blocks NASH development in BI-1-deficient mice.

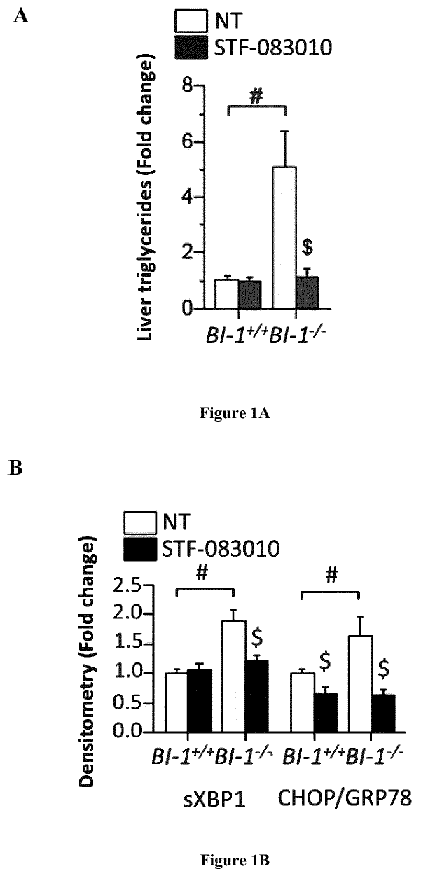

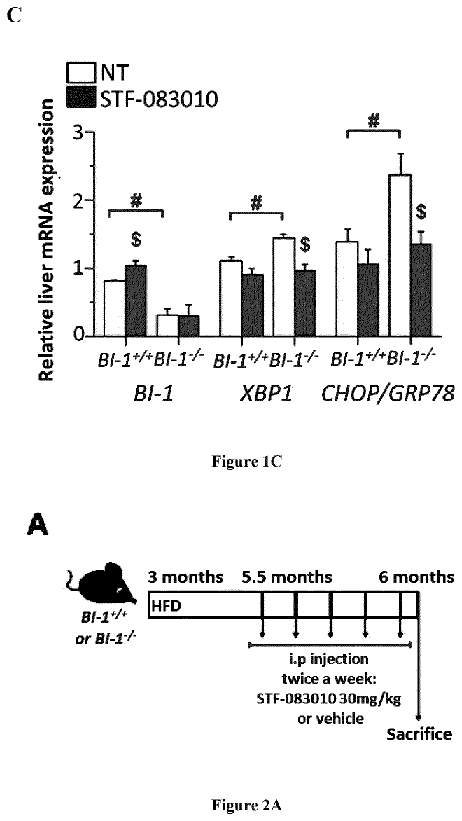

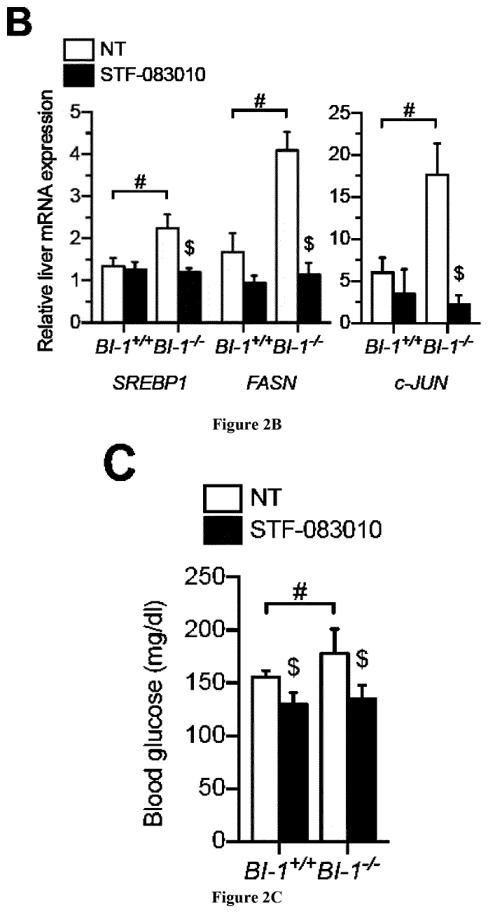

To establish a therapeutic window for targeting IRE1α in vivo, we evaluated the kinetics of HFD-induced liver disease in BI-1^{-/-} mice and found that short-term HFD feeding leads to significantly greater weight gain (that normalizes after long-term HFD detailed above) and higher ALT levels in BI-1^{-/-} mice (FIG. 2A-C). By 3 months of HFD, BI-1^{-/-} mice already exhibit hyperglycemia from impaired glucose tolerance and reduced insulin sensitivity as compared to WT mice. As BI-1^{-/-} mice develop the first signs of NAFLD between 2 and 3 months after initiation of HFD, we administered STF-083010 during this time period concomitantly with HFD (FIG. 2A). Macroscopically, targeting IRE1α RNase function prevented liver discoloration seen in BI-1^{-/-} mice fed a HFD for 3 months. Microscopically in BI-1^{-/-} HFD mice, treatment with STF-083010 suppressed histological signs of inflammation and grade-3 steatosis. Normalized liver triglyceride content in HFD-fed BI-1^{-/-} STF-083010 mice (FIG. 1A) was found to be due to a correction in lipid droplet hypertrophy, shifting lipid droplet diameter from massive to average size in hepatocytes. Metabolic master regulators (SREBP1, FASN and c-JUN) were upregulated in NT HFD-fed BI-1^{-/-} mice but within normal mRNA levels in STF-083010-treated mice (FIG. 2B). Inhibiting IRE1α RNase function led to a normalization of blood glucose concentrations in HFD-fed mice (FIG. 2C). In livers from BI-1^{-/-} mice, STF-083010 reduced the expression of IRE1α RNase target sXBP1 and the deleterious CHOP/GRP78 ratio at the protein and mRNA levels. In WT mice fed a HFD, STF-083010 treatment induced a 15% increase in BI-1 mRNA levels that possibly reinforce the beneficial impact of targeting IRE1α RNase activity (FIG. 1C).

STF-083010 treatment counteracted the activation of caspase-1/-11 and NLRP3 proteins in BI-1^{-/-} HFD-fed mice and normalized the mRNA levels of NLRP3 inflammasome and pro-inflammatory response components. A significant decrease in MPO-positive cells was also apparent between untreated and STF-083010-treated mice fed a HFD. Limiting IRE1α RNase activity in HFD-fed mice protected from liver injury in both genotypes. STF-083010 treatment prevented the collagen accumulation with cellular infiltration seen in BI-1^{-/-} mice after 3 months of HFD and significantly limited hepatic Col6a3 upregulation. In ND-fed mice, STF-083010 treatment did not alter the appearance of the liver. These data indicate that STF-083010 reverses the propensity of BI-1-deficient mice to develop NASH.

Inhibition of IRE1α signaling enhances the effectiveness of current chemotherapy in HCC.

We observed that the IRE1α RNase is constitutively activated in HCC cell lines and in HCC liver biopsies contributing to the basal production of pro-tumorigenic factors (data not shown). Indeed HCC cell lines and HCC liver biopsies exhibit a constitutive IRE1α signaling and a BI-1 downregulation.

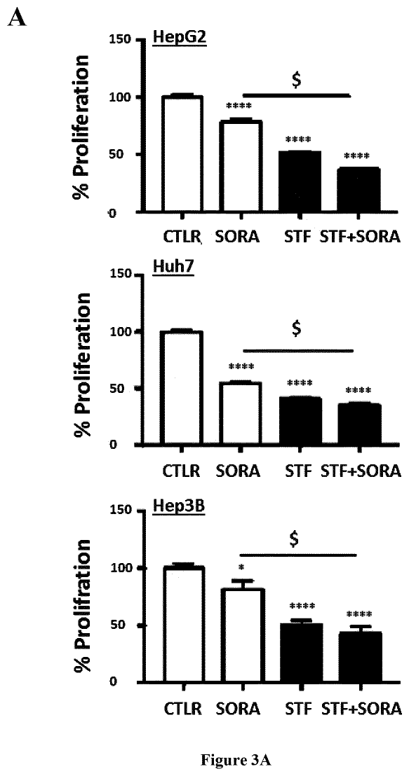

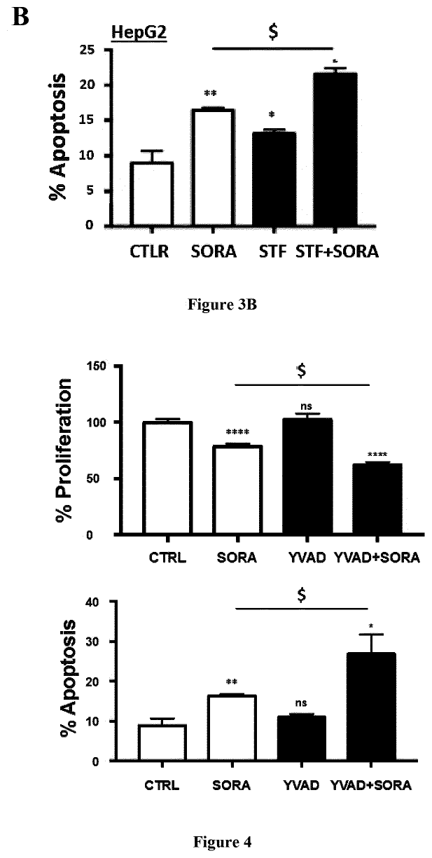

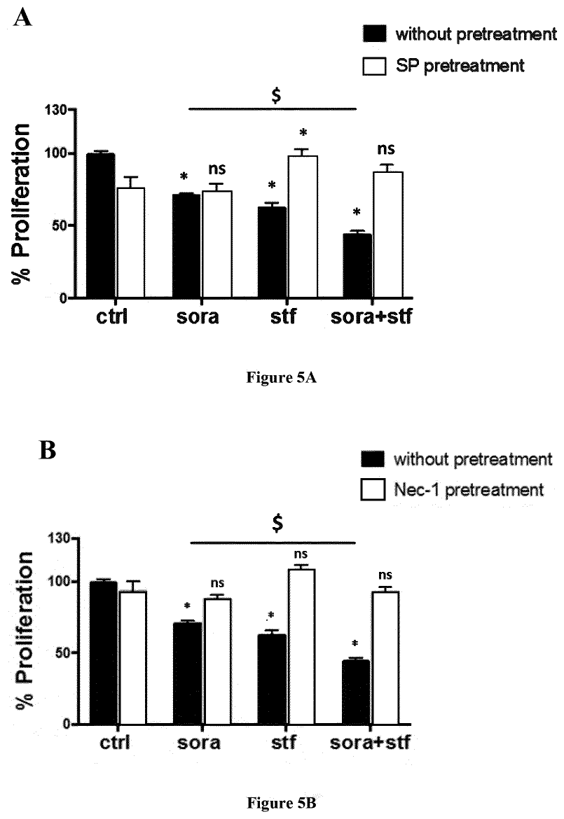

The IRE1α RNase activity inhibitor SFT-083010 reduces liver cancer cell proliferation (FIG. 3A). When SFT-083010 was co-treated with sorafenib, it improves their effectiveness in reducing liver cancer cell proliferation (FIG. 3A) and inducing apotosis in HCC cell lines (FIG. 3B). These data were confirmed with MKC6688 and 4μ8c, other inhibitors of IRE1α RNase activity (data not shown). We observed that the hyperactive of IRE1α RNase triggers the secretion of pro-inflammatory markers such as caspase-1, IL-1β, IL-6 (data not shown). Ac-YVAD-CMK, a caspase-1/-11 inhibitor enhances also the effectiveness of sorafenib in reducing liver cancer cell proliferation and inducing apoptosis, however not more efficiently than SFT-083010 (FIG. 4). Necrostatin-1 (Nec-1), a RIPK1 kinase inhibitor, and SP600125 (SP), a JNK inhibitor, seem to inhibit the effect of the Sorafenib and SFT-083010. After a Nec-1 or SP pre-treatment, cancer cell proliferation is similar with or without injection of sorafenib and/or SFT-083010 (FIGS. 5A and 5B). The co-treatment of sorafenib and STF-083010 enhances apoptosis via the kinase activity of IRE1α.

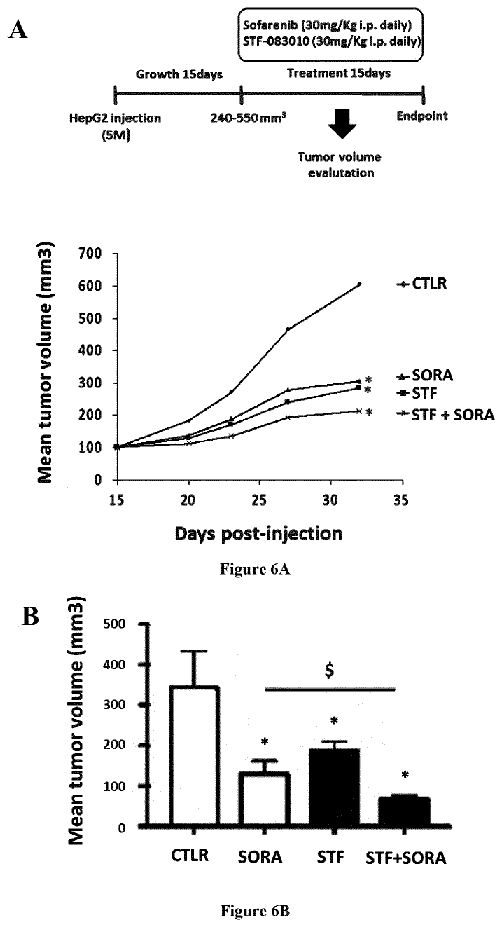

In a xenograft mouse model of HCC, inhibition of IRE1α RNase activity with STF-083010 increased sorafenib-mediated tumor suppression (FIGS. 6A and 6B). We confirmed the data with MKC6688, the last generation inhibitor of IRE1α RNase activity (data not shown), suggesting that inclusion of IRE1α RNase inhibitors could enhance the effectiveness of current chemotherapy.

### REFERENCES

Throughout this application, various references describe the state of the art to which this invention pertains. The disclosures of these references are hereby incorporated by reference into the present disclosure.
1. Younossi Z M, Blissett D, Blissett R, Henry L, Stepanova M, Younossi Y, Racila A, Hunt S, Beckerman R. The economic and clinical burden of nonalcoholic fatty liver disease in the United States and Europe. Hepatology 2016; 64:1577-1586.
2. Angulo P. Nonalcoholic fatty liver disease. N Engl J Med 2002; 346:1221-31.
3. Walter P, Ron D. The unfolded protein response: from stress pathway to homeostatic regulation. Science 2011; 334:1081-6.
4. Hetz C. The unfolded protein response: controlling cell fate decisions under ER stress and beyond. Nat Rev Mol Cell Biol 2012; 13:89-102.
5. Ozcan U, Cao Q, Yilmaz E, Lee A H, Iwakoshi N N, Ozdelen E, Tuncman G, Gorgun C, Glimcher L H, Hotamisligil G S. Endoplasmic reticulum stress links obesity, insulin action, and type 2 diabetes. Science 2004; 306:457-61.
6. Ozcan U, Yilmaz E, Ozcan L, Furuhashi M, Vaillancourt E, Smith R O, Gorgun C Z, Hotamisligil G S. Chemical chaperones reduce ER stress and restore glucose homeostasis in a mouse model of type 2 diabetes. Science 2006; 313:1137-40.
7. Nakatani Y, Kaneto H, Kawamori D, Yoshiuchi K, Hatazaki M, Matsuoka T A, Ozawa K, Ogawa S, Hori M, Yamasaki Y, Matsuhisa M. Involvement of endoplasmic reticulum stress in insulin resistance and diabetes. J Biol Chem 2005; 280:847-51.
8. Gregor M F, Yang L, Fabbrini E, Mohammed B S, Eagon J C, Hotamisligil G S, Klein S. Endoplasmic reticulum stress is reduced in tissues of obese subjects after weight loss. Diabetes 2009; 58:693-700.
9. Hotamisligil G S. Endoplasmic reticulum stress and the inflammatory basis of metabolic disease. Cell 2010; 140:900-17.
10. Han J, Kaufman R J. The role of ER stress in lipid metabolism and lipotoxicity.

J Lipid Res 2016; 57:1329-38.
11. Brenner C, Galluzzi L, Kepp O, Kroemer G. Decoding cell death signals in liver inflammation. J Hepatol 2013; 59:583-94.
12. Sharma D, Kanneganti T D. The cell biology of inflammasomes: Mechanisms of inflammasome activation and regulation. J Cell Biol 2016; 213:617-29.
13. Wree A, Eguchi A, McGeough M D, Pena C A, Johnson C D, Canbay A, Hoffman H M, Feldstein A E. NLRP3 inflammasome activation results in hepatocyte pyroptosis, liver inflammation and fibrosis. Hepatology 2013.
14. Man S M, Kanneganti T D. Converging roles of caspases in inflammasome activation, cell death and innate immunity. Nat Rev Immunol 2016; 16:7-21.
15. Lebeaupin C, Proics E, de Bieville C H, Rousseau D, Bonnafous S, Patouraux S, Adam G, Lavallard V J, Rovere C, Le Thuc O, Saint-Paul M C, Anty R, Schneck A S, Iannelli A, Gugenheim J, Tran A, Gual P, Bailly-Maitre B. ER stress induces NLRP3 inflammasome activation and hepatocyte death. Cell Death Dis 2015; 6:e1879.
16. Bailly-Maitre B, Belgardt B F, Jordan S D, Coornaert B, von Freyend M J, Kleinridders A, Mauer J, Cuddy M, Kress C L, Willmes D, Essig M, Hampel B, Protzer U, Reed J C, Bruning J C. Hepatic Bax inhibitor-1 inhibits IRE1alpha and protects from obesity-associated insulin resistance and glucose intolerance. J Biol Chem 2010; 285:6198-207.
17. Chae H J, Kim H R, Xu C, Bailly-Maitre B, Krajewska M, Krajewski S, Banares S, Cui J, Digicaylioglu M, Ke N, Kitada S, Monosov E, Thomas M, Kress C L, Babendure J R, Tsien R Y, Lipton S A, Reed J C. BI-1 regulates an apoptosis pathway linked to endoplasmic reticulum stress. Mol Cell 2004; 15:355-66.
18. Bailly-Maitre B, Fondevila C, Kaldas F, Droin N, Luciano F, Ricci J E, Croxton R, Krajewska M, Zapata J M, Kupiec-Weglinski J W, Farmer D, Reed J C. Cytoprotective gene bi-1 is required for intrinsic protection from endoplasmic reticulum stress and ischemia-reperfusion injury. Proc Natl Acad Sci USA 2006; 103:2809-14.
19. Lisbona F, Rojas-Rivera D, Thielen P, Zamorano S, Todd D, Martinon F, Glavic A, Kress C, Lin J H, Walter P, Reed J C, Glimcher L H, Hetz C. BAX inhibitor-1 is a negative regulator of the ER stress sensor IRE1alpha. Mol Cell 2009; 33:679-91.
20. Calfon M, Zeng H, Urano F, Till J H, Hubbard S R, Harding H P, Clark S G, Ron D. IRE1 couples endoplasmic reticulum load to secretory capacity by processing the XBP-1 mRNA. Nature 2002; 415:92-6.
21. Maurel M, Chevet E, Tavernier J, Gerlo S. Getting RIDD of RNA: IRE1 in cell fate regulation. Trends Biochem Sci 2014; 39:245-54.
22. Lee A H, Scapa E F, Cohen D E, Glimcher L H. Regulation of hepatic lipogenesis by the transcription factor XBP1. Science 2008; 320:1492-6.
23. Patouraux S, Rousseau D, Rubio A, Bonnafous S, Lavallard V J, Lauron J, Saint-Paul M C, Bailly-Maitre B, Tran A, Crenesse D, Gual P. Osteopontin deficiency aggravates hepatic injury induced by ischemia-reperfusion in mice. Cell Death Dis 2014; 5:e1208.
24. Flodby P, Barlow C, Kylefjord H, Ahrlund-Richter L, Xanthopoulos K G. Increased hepatic cell proliferation and lung abnormalities in mice deficient in CCAAT/enhancer binding protein alpha. J Biol Chem 1996; 271:24753-60.
25. Brydges S D, Mueller J L, McGeough M D, Pena C A, Misaghi A, Gandhi C, Putnam C D, Boyle D L, Firestein G S, Homer A A, Soroosh P, Watford W T, O'Shea J J, Kastner D L, Hoffman H M. Inflammasome-mediated disease animal models reveal roles for innate but not adaptive immunity. Immunity 2009; 30:875-87.
26. Rensen S S, Slaats Y, Nijhuis J, Jans A, Bieghs V, Driessen A, Malle E, Greve J W, Buurman W A. Increased hepatic myeloperoxidase activity in obese subjects with nonalcoholic steatohepatitis. Am J Pathol 2009; 175:1473-82.
27. Robert S, Gicquel T, Victoni T, Valenca S, Barreto E, Bailly-Maitre B, Boichot E, Lagente V. Involvement of matrix metalloproteinases (MMPs) and inflammasome pathway in molecular mechanisms of fibrosis. Biosci Rep 2016; 36.
28. Oyadomari S, Mori M. Roles of CHOP/GADD153 in endoplasmic reticulum stress. Cell Death Differ 2004; 11:381-9.
29. Csak T, Ganz M, Pespisa J, Kodys K, Dolganiuc A, Szabo G. Fatty acid and endotoxin activate inflammasomes in mouse hepatocytes that release danger signals to stimulate immune cells. Hepatology 2011; 54:133-44.
30. Papandreou I, Denko N C, Olson M, Van Melckebeke H, Lust S, Tam A, Solow-Cordero D E, Bouley D M, Offner F, Niwa M, Koong A C. Identification of an Irelalpha endonuclease specific inhibitor with cytotoxic activity against human multiple myeloma. Blood 2011; 117:1311-4.
31. Kriss C L, Pinilla-Ibarz J A, Mailloux A W, Powers J J, Tang C H, Kang C W, Zanesi N, Epling-Burnette P K, Sotomayor E M, Croce C M, Del Valle J R, Hu C C. Overexpression of TCL1 activates the endoplasmic reticulum stress response: a novel mechanism of leukemic progression in mice. Blood 2012; 120:1027-38.
32. Tufanli O, Telkoparan Akillilar P, Acosta-Alvear D, Kocaturk B, Onat U I, Hamid S M, Cimen I, Walter P, Weber C, Erbay E. Targeting IRE1 with small molecules counteracts progression of atherosclerosis. Proc Natl Acad Sci USA 2017; 114:E1395-E1404.
33. Ron D, Brasier A R, McGehee R E, Jr., Habener J F. Tumor necrosis factor-induced reversal of adipocytic phenotype of 3T3-L1 cells is preceded by a loss of nuclear CCAAT/enhancer binding protein (C/EBP). J Clin Invest 1992; 89:223-33.
34. Villacorta L, Garcia-Barrio M T, Chen Y E. Transcriptional regulation of peroxisome proliferator-activated receptors and liver X receptors. Curr Atheroscler Rep 2007; 9:230-7.
35. Rutkowski D T, Wu J, Back S H, Callaghan M U, Ferris S P, Iqbal J, Clark R, Miao H, Hassler J R, Fornek J, Katze M G, Hussain M M, Song B, Swathirajan J, Wang J, Yau G D, Kaufman R J. UPR pathways combine to prevent hepatic steatosis caused by ER stress-mediated suppression of transcriptional master regulators. Dev Cell 2008; 15:829-40.
36. Xu C, Xu W, Palmer A E, Reed J C. BI-1 regulates endoplasmic reticulum Ca2+ homeostasis downstream of Bcl-2 family proteins. J Biol Chem 2008; 283:11477-84.
37. Bailly-Maitre B, Bard-Chapeau E, Luciano F, Droin N, Bruey J M, Faustin B, Kress C, Zapata J M, Reed J C. Mice lacking bi-1 gene show accelerated liver regeneration. Cancer Res 2007; 67:1442-50.
38. Lerner A G, Upton J P, Praveen P V, Ghosh R, Nakagawa Y, Igbaria A, Shen S, Nguyen V, Backes B J, Heiman M, Heintz N, Greengard P, Hui S, Tang Q, Trusina A, Oakes S A, Papa F R. IRE1alpha induces thioredoxin-interacting protein to activate the NLRP3 inflammasome and promote programmed cell death under irremediable E R stress. Cell Metab 2012; 16:250-64.
39. Mridha A R, Wree A, Robertson A A, Yeh M M, Johnson C D, Van Rooyen D M, Haczeyni F, Teoh N C, Savard C, Ioannou G N, Masters S L, Schroder K, Cooper M A, Feldstein A E, Farrell G C. NLRP3 inflammasome blockade reduces liver inflammation and fibrosis in experimental NASH in mice. J Hepatol 2017.
40. Hernandez-Gea V, Hilscher M, Rozenfeld R, Lim M P, Nieto N, Werner S, Devi L A, Friedman S L. Endoplasmic reticulum stress induces fibrogenic activity in hepatic stellate cells through autophagy. J Hepatol 2013; 59:98-104.
41. Kim R S, Hasegawa D, Goossens N, Tsuchida T, Athwal V, Sun X, Robinson C L, Bhattacharya D, Chou H I, Zhang D Y, Fuchs B C, Lee Y, Hoshida Y, Friedman S L. The XBP1 Arm of the Unfolded Protein Response Induces Fibrogenic Activity in Hepatic Stellate Cells Through Autophagy. Sci Rep 2016; 6:39342.

1. An inhibitor of the endoribonuclease activity of IRE1α for use for treating a subject suffering from a liver disease wherein, the inhibitor of endoribonuclease activity of IRE1α is MKC-8866.

2. The inhibitor of the endoribonuclease activity of IRE1α for use according to claim 1, wherein the liver disease is selected from the group consisting of: nonalcoholic fatty liver disease (NAFLD), non-alcoholic steatohepatitis (NASH), fibrotic NASH or liver cancer.

3. The inhibitor of the endoribonuclease activity of IRE1α for use according to claim 1, wherein the inhibitor of endoribonuclease activity of IRE1α is administered in combination with a classical treatment of a liver disease, wherein the classical treatment is sorafenib.
